(19) United States
(12) Patent Application Publication
(10) Pub. No.: US 2022/0139250 A1
(43) Pub. Date: May 5, 2022

(54) Method and System to generate a fillable digital scape (FDS) to reinforce mind integration

(71) Applicant: Loona Inc., Dover, DE (US)

(72) Inventors: Andrei Yanchurevich, Minsk (BY); Dzmitry Doryn, Minsk (BY); Mikhail Charniatsou, Minsk (BY); Siarchei Hanchar, London (GB); Eugene Nevgen, Minsk (BY)

(21) Appl. No.: 17/087,148

(22) Filed: Nov. 2, 2020

(51) Int. Cl. G09B 19/00; G06F 3/16; G06N 20/00; A61B 5/00; A61B 5/16

(52) U.S. Cl. CPC G09B 19/00; G06F 3/165; G06N 20/00; G06F 3/04883; A61B 5/165; A61B 5/681; A61B 5/4815

(57) ABSTRACT

Disclosed is a method and system for generating a fillable digital scape (FDS) for mind integration. The invention comprises a method of pushing the FDS, interacting with the FDS with a guided prompt; matching precisely with the guided prompt to create a digital event and tracking progress of the filling of the FDS until completion. Additionally, the FDS is pushed based on at least one of age, sleep schedule or quality of sleep, current, pre-existing or chronic mental or somatic condition, musical, graphical, color, personal preferences and a stress level.

### TECHNICAL FIELD

This invention relates generally to the field generating a fillable digital scape (FDS) to reinforce mind integration. More specifically, the invention discloses a new and useful method for generating a sleep sanctuary based on the user's emotional, physical or mental state.

### BACKGROUND

Sleep is a naturally recurring state of mind and body, characterized by altered consciousness, relatively inhibited sensory activity, reduced muscle activity and inhibition of nearly all voluntary muscles during rapid eye movement (REM) sleep and reduced interactions with surroundings. It is distinguished from wakefulness by a decreased ability to react to stimuli, but more reactive than a coma or disorders of consciousness, with sleep displaying quite different and active brain patterns. Sleep supports emotional, physiological and reparative regeneration. It is known to affect a person's quality of life and well-being. There are several factors able to affect regular sleep: age, gender, environment, psychological state, illnesses and the use of medication.

During sleep, most of the body systems or in the end an about state, helping to restore the immune, nervous, skeletal, and muscular system; these are vital processes that maintain mood, memory, and cognitive function, and play a large role in the function of the endocrine and immune systems. The internal circadian clock promotes sleep daily at night. The diverse purposes and mechanisms of sleep are the subject of substantial ongoing research. Sleep is a highly conserved behavior across animal evolution

Humans may suffer from various sleep disorders, including dyssomnias such as insomnia, hypersomnia, narcolepsy, and sleep apnea; parasomnias such as sleep walking and rapid eye movement sleep behavior disorder; bruxism; and circadian rhythm sleep disorders. Insomnia is a general term for difficulty falling asleep and/or staying asleep. Insomnia is the most common sleep problem, with many adults reporting occasional insomnia, and 10-15% reporting a chronic condition. Insomnia can have many different causes, including psychological stress, a poor sleep environment, an inconsistent sleep schedule, or excessive mental or physical stimulation in the hours before bedtime. Insomnia is often treated through behavioral changes like keeping a regular sleep schedule, avoiding stimulating or stressful activities before bedtime, and cutting down on stimulants such as caffeine. The sleep environment may be improved by installing heavy drapes to shut out all sunlight, and keeping computers, televisions and work materials out of the sleeping area.

Currently insomnia may also be due to hyperarousal experienced within the day which may lead to difficulty initiating and maintaining sleep at night. This process is explained by the cognitive model which states that worry and concerns due to usual daily stress affect sleep, leading to onsets of insomnia. Usually, it manifests mostly with problems in initiating sleep and returning back to sleep after an awakening. Since a person begins to experience sleep difficulties, worry and concerns change their focus from usual stress to worries about insomnia. This negative cognitive activity becomes only worse when exacerbated by the sleep deficit.

Proper stress management is crucial in the insomnia treatment approaches, as evidence shows that relaxation exercises may improve sleep quality by relaxing the body, lowering blood pressure, stimulating circulation, and relaxing the muscles. Relaxation exercises are based on reducing fear, excitement, anxiety, palpitations, and spasms, as well as slowing thought. They are useful to create a calm, comfortable and relaxed response. Provision of the digital software for calming, relaxation, and mindfulness interventions has the potential to become a part of ongoing self-care. Time-bounded sessions in the app each has its specific aim to help control the pre-sleep screen time, reinforce relaxation and instill better sleep hygiene.

Cognitive-behavioral symptom management strategies indicate beneficial effects of relaxation, meditation, and guided imagery. Imagery is the thought process that takes into account and translates the senses for the nervous system to, in turn, produce healing change throughout the body. Guided imagery involves external instructional guidance to allow the internal generation of images, which invoke visual, auditory, haptic and taste-smell experiences as well triggering behavioral and physiological responses. Guided imagery may be done through prompting by a live practitioner or an audio record. Imagery induces a deep sense of calm and an avenue of reframing emotions and thus changing the balance of the nervous system. A neuroanatomic model suggests that nonverbal images and the processing of emotions occur in the right cerebral hemisphere. This association is translated into an autonomic response. The left hemisphere's conscious control of the voluntary nervous system then modulates the emotional-autonomic response pathway. Guided imagery can profoundly activate the body's natural relaxation response as characterized by slower breathing and lower blood pressure as well as increased feelings of well-being. During the process of meditation, accumulated stresses are removed, energy is increased, and health is positively affected overall. Research has confirmed a myriad of health benefits associated with the practice of meditation by generating relaxing states through mental processes. These include stress reduction, decreased anxiety and depression symptoms, reduction in pain (both physical and psychosomatic), improving sleep quality. As a treatment intervention, there is a strong evidence base for guided imagery in stress and anxiety management in different populations. Physiological benefits include reduced blood pressure, heart rate, and stress hormones (cortisol and epinephrine), and increased melatonin (sleep hormone).

Art activity is used to improve cognitive and sensorimotor functions, foster self-esteem and self-awareness, cultivate emotional resilience, promote insight, enhance social skills, reduce and resolve conflicts and distress, and advance societal and ecological change. Art activity is a way for these troubled persons to feel expressive in a non-judgmental environment. This may be due to certain therapeutic mechanisms that researchers assume to be relevant: hedonism and play, aesthetic experience and authenticity, nonverbal communication and symbolizing, test-acting in an inactive transitional space, creation and generativity. Creative arts interventions and craft activity were found to have a positive impact on perceived stress and stress management. Art techniques were effective in reduction of anxiety levels, improved the subjects' mood and had a positive impact on the symptoms of depression. Art activity leads to improvement of general well-being through the reduction of negative and the enhancement of positive emotions, contributes to the improvement of overall health and well-being by distracting individuals from intrusive thoughts and improves self-identity. It is effective in reduction of perceived symptoms related to psychosomatic disorders. Art activity was found to be helpful in coping with professional burnout. Creative self-expression lowers cortisol (stress hormone) levels. Art making with using brushes and paint to decorate a tile decreases cortisol levels and helps to reduce feelings of sluggishness and improve concentration.

Music can be considered a safe and generally well-accepted individualized intervention provided in a person-centered way to alleviate symptoms of stress and improve quality of life. The restorative effect of music has been recognized for many years. In the 16th and 17th centuries, music was used to treat mood disorders. To date, the use of music for therapeutic purposes has become prevalent worldwide, and its effect on various mental disorders has been studied. Effectiveness of musical interventions was proved in improving mood, symptoms of depression, quality of life, global and social functioning, functional recovery, and neuromotor performances. Significant effects of music listening on sleep quality have been found in various populations. Sleep improvements were assessed by polysomnography. Music in itself can be effective in improving sleep and music influence with additional interventions like imagery, guided relaxation procedure or progressive muscle relaxation significantly improve sleep quality.

In a national sleep survey, 40 percent of respondents said they aren't getting the recommended amount of rest. Many of the stressors we face in modern life, such as traffic jams, difficult co-workers, or relationship conflicts, can trigger a fight-or-flight response, and prolonged exposure to this stress without relaxation can result in shorter sleep duration and poorer quality sleep. The CDC (Center for Disease Control and Prevention) calls this situation an epidemic—“Sleep and sleep disorders, such as obstructive sleep apnea and insomnia, are increasingly recognized as vital to a wide variety of public health and chronic disease concerns, including obesity, hypertension and cancer,” said Janet B. Croft, PhD, CDC senior chronic disease epidemiologist in the Division of Population Health.

With this kind of stressful life, it is becoming readily apparent that a solution is desperately needed in order to prevent this epidemic from taking over our daily lives. There is a void for a solution that takes into account an individualistic personalized approach based on a user's physical, emotional and mental state. These effects will only logarithmically grow as we transition into the IoT era—where we will be exposed to thousands of internet-enabled objects (each capable of delivering contextualized analytics and provisioning) as part of our day-to-day living.

### SUMMARY

Disclosed is a method and system for generating a fillable digital scape (FDS) for mind integration. In one embodiment of the invention, the method comprises: pushing the FDS, interacting with the FDS with a guided prompt; matching precisely with the guided prompt to create a digital event and tracking progress of the filling of the FDS until completion. Further yet, the FDS is pushed based on at least one of age, sleep schedule or quality of sleep, current, pre-existing or chronic mental or somatic condition, musical, graphical, color, personal preferences and a stress level. Additionally, in an embodiment of the invention, the FDS is selected for a user from a plurality of said FDS, the FDS indicating an assessment of at least one of a feeling, sensation, mood, mental, emotional or physical state of the user. The assessment of the FDS is based on at least one of a standard Pittsburg sleep quality index (PSQI), consensus sleep diary (CSD), daytime insomnia scale (DISS), insomnia symptom questionnaire (ISQ), Pittsburg insomnia rating scale (PIRS), a smartwatch, a ring, a bracelet, a necklace, a body-worn device and IoT devices.

In another embodiment of the invention, the method for generating a FDS for mind integration comprises: selecting the FDS by a user based on at least one of a feeling, sensation, mood, mental, emotional or physical state; interacting with the FDS with a guided prompt; matching precisely with the guided prompt to create a digital event; and tracking progress of the filling of the FDS until completion. Further yet, in an embodiment of the invention the interaction with the FDS is at least one of a touch, swipe, feel, pull, push, drag, pinch, gesture, multi-touch gestures and natural user interface techniques. Additionally, in an embodiment of the invention, the guided prompt is at least one of textual, video, audio, visual, gestural, verbal, physical, vibration, phonemic and positional. In yet another embodiment of the invention, the digital event is at least one of a meditation, art, color, digital coloring and music to progressively fill the FDS.

Further yet, in an embodiment of the invention, the method of generating a FDS for mind integration comprises the steps of: selecting the FDS for a user based on at least one of a feeling, sensation, mood, mental, emotional or physical state of the user; pushing the FDS based on at least one of age, sleep schedule or quality of sleep, current, pre-existing or chronic mental or somatic condition, musical, graphical, color, personal preferences and a stress level; interacting with the FDS with a guided prompt; matching precisely with the guided prompt to create a digital event and tracking progress of the filling of the FDS until completion.

Further yet, in an embodiment of the present invention, a method for generating a fillable digital scape (FDS) for mind integration comprises the steps of: selecting the FDS for a user based on at least one of a feeling, sensation, mood, mental, emotional or physical state of the user, pushing the FDS based on at least one of age, sleep schedule or quality of sleep, current, pre-existing or chronic mental or somatic condition, musical, graphical, color, personal preferences and a stress level, interacting with the FDS with a guided prompt, wherein the guided prompt is at least one of textual, video, audio, visual, gestural, verbal, physical, vibration, phonemic and positional, repeating the interactions with the FDS in a steady progressive method to create a digital experience, wherein the digital experience is at least one of a meditation, art, color, digital coloring and music; and tracking progress of the filling of the FDS until completion to achieve mind integration.

Further yet, in an embodiment of the invention, the FDS assessment is based on at least one of a standard Pittsburg sleep quality index (PSQI), consensus sleep diary (CSD), daytime insomnia scale (DISS), insomnia symptom questionnaire (ISQ), Pittsburg insomnia rating scale (PIRS), a smartwatch, a ring, a bracelet, a necklace, a body-worn device and IoT devices.

Additionally, in an embodiment of the invention, the interaction with the FDS is at least one of touch, swipe, feel, pull, push, drag, pinch, gesture, multi-touch gestures and natural user interface techniques. Further yet, the completion of the FDS is via a positive affirmation. The positive affirmation is at least one of continuing with another FDS session or based on a time triggered event. The method of generating the FDS is further comprised of establishing a behavioral intervention to target sleep directly or improve sleep hygiene routine.

For example, in one application of the invention, a specific FDS—a tepee in the wilderness is selected by the user based on the user's emotional, mental and physical state. The invention can classify this FDS based on the user's personal preferences for the love of rough country and nomadic lifestyle—Gen Z kid who loves technology. The user's preference may differ on the emotional, current, preexisting or chronic mental or somatic condition at one time. The user may change their preference at any time.

In another object of the invention, the assessment of the FDS is based on at least one of a standard Pittsburg sleep quality index (PSQI), consensus sleep diary (CSD), daytime insomnia scale (DISS), insomnia symptom questionnaire (ISQ), Pittsburg insomnia rating scale (PIRS), a smartwatch, a ring, a bracelet, a necklace, a body-worn device and IoT devices. Additionally, in another embodiment of the invention, the guided activity is at least one of a meditation, art, color, digital coloring and music.

Additionally, for example, in one application of the invention, a specific FDS—tropical forest with sounds of falling rain is selected for a user based on a user's emotional, mental or physical state. The invention can classify this FDS based on the user's age—mid 40's, with one infant—lack/poor quality of sleep, PSQI sleep score, loves ocean/water, that the user will tend to relax and instill calmness after a long day of physical work and poor quality of sleep.

In an embodiment of the invention, the FDS is at least one of a black & white or a colored, a 2D or a 3D diorama. Additionally, the dioramas are at least one of varying sizes, a miniature or a large-scale object showing a plurality of details. Further yet, in an embodiment, the dioramas are a plurality of mountains, oceans, past memories, landscapes, cities, objects, historical events, ecological biomes, cultural scenes, visually depicted literature, ships, buildings, vehicles, railways, architectural and historical venues and entertainment or museums. Moreover, the dioramas may be either static or dynamic.

In yet another embodiment of the invention, a system for generating a fillable digital scape (FDS) to reinforcing mind integration comprising of: a FDS classifier; a FDS selector; a FDS prescriber; a non-transitory storage element coupled to the processor, encoded instructions stored in the non-transitory storage element, wherein the encoded instructions when implemented by the processor, configure the sleep sanctuary channel to: classify the FDS based on at least one of age, sleep schedule or quality of sleep, current, pre-existing or chronic mental or somatic condition, musical, graphical, color, personal preferences and a stress level by the FDS classifier; select at least one of the FDS for the user from a plurality of the FDS by assessing at least one of a feeling, sensation, mood, mental, emotional or physical state of the user via the FDS selector; push the FDS via the FDS prescriber personalized to the user based on at least one stored message coupled to the FDS, wherein said message comprises of a guided prompt to interact with the FDS to create a digital event, wherein the digital event is at least one of a meditation, art, color, digital coloring and music to progressively fill the FDS and track the progress of the filling of the FDS until completion.

Whether the FDS are generated by the system or defined by the user, content is being overlaid or delivered to enhance relaxation, boost mood, support self-esteem, inspire wellness, and aid in the longitudinal and non-interventional care for people in distress or need—leveraging a familiar and known modality (digital devices). According to the claimed invention, a whole ecosystem of receiving and delivering modalities are provided for a host of digital therapeutics. The digital therapeutic offerings—with the aid of Artificial Intelligence (AI), machine learning, and, or predictive emotional and mental assessment tools—may deliver increasingly personalized solutions uniquely tailored to aid each subscriber/user. Such non-interventional, anonymous, and device-centric solutions are far more appropriate to combat the rising ill-effects of everyday stresses—rather than pharmaceutical dosing, in-patient treatment, and altering device behavior.

### BRIEF DESCRIPTION OF DRAWINGS

FIG. 1 illustrates a representative method flow of the fillable digital scape (FDS) in accordance with an aspect of the invention.

FIG. 2 illustrates a representative block diagram of the FDS in accordance with an aspect of the invention.

FIG. 3 illustrates a representative interaction flow of the FDS in accordance with an aspect of the invention.

FIG. 4A-D illustrates a series of progression of FDS in accordance with an aspect of the invention.

FIG. 5A-C illustrates a series of completed FDS in accordance with an aspect of the invention.

FIG. 6 illustrates a network diagram in accordance with an aspect of the invention.

FIG. 7 illustrates a representative block diagram of the fillable digital scape system in accordance with an aspect of the invention.

FIG. 8 illustrates a representative interaction flow of the fillable digital scape system in accordance with an aspect of the invention.

### DETAILED DESCRIPTION OF DRAWINGS

Numerous embodiments of the invention will now be described in detail with reference to the accompanying figures. The following description of the embodiments of the invention is not intended to limit the invention to these embodiments but rather to enable a person skilled in the art to make and use this invention. Variations, configurations, implementations, and applications described herein are optional and not exclusive to the variations, configurations, implementations, and applications they describe. The invention described herein can include any and all permutations of these variations, configurations, implementations, and applications.

In the following description, numerous specific details are set forth in order to provide a thorough understanding of the invention. It will be apparent, however, to one skilled in the art that the invention can be practiced without these specific details.

Reference in this specification to “one embodiment” or “an embodiment” means that a particular feature, structure, or characteristic described in connection with the embodiment is included in at least one embodiment of the invention. The appearances of the phrase “in one embodiment” in various places in the specification are not necessarily all referring to the same embodiment, nor are separate or alternative embodiments mutually exclusive of other embodiments. Moreover, various features are described which may be exhibited by some embodiments and not by others. Similarly, various requirements are described which may be requirements for some embodiments but no other embodiments.

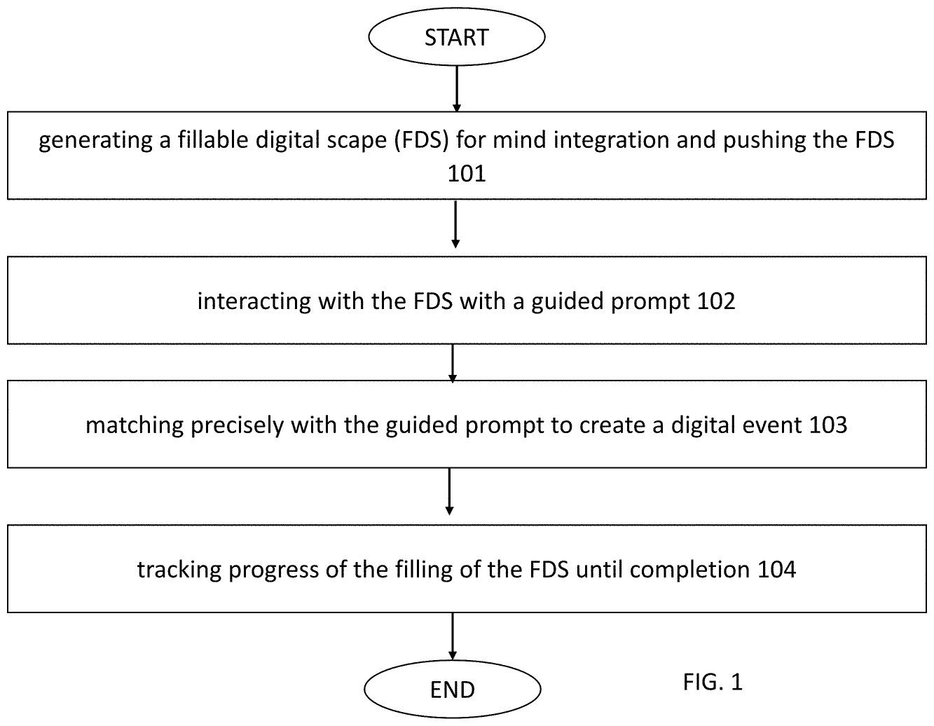

With reference to FIG. 1, in an embodiment of the invention, a method for generating a fillable digital scape (FDS) for mind integration, comprising the steps of pushing the FDS 101; interacting with the FDS with a guided prompt 102; matching precisely with the guided prompt to create a digital event 103 and tracking progress of the filling of the FDS until completion 104. Additionally, in an embodiment of the invention, the pushing of the FDS is based on at least one of age, sleep schedule or quality of sleep, current, pre-existing or chronic mental or somatic condition, musical, graphical, color, personal preferences and a stress level.

The FDS may be generated on any electronic computing device with a processor which is communicatively coupled via a communication network. The network may be any class of wired or wireless network including any software, hardware, or computer applications that can provide a medium to exchange signals or data. The network may be a local, regional, or global communication network.

The electronic computing device may be any electronic device capable of sending, receiving, and processing information. Examples of the computing device include, but are not limited to, a smartphone, a mobile device/phone, a Personal Digital Assistant (PDA), a computer, a workstation, a notebook, a mainframe computer, a laptop, a tablet, a smart watch, an internet appliance and any equivalent device capable of processing, sending and receiving data. Additionally, the user may also be connected to augmented reality (AR) or virtual reality (VR) headset capable of performing an electroencephalograph (EEG) or an equivalent brain mapping technique. The headset can monitor and record these thoughts and feelings using the EEG and transcribe the thoughts and feelings into an electronic message or send the EEG output signals directly to a device system. The electronic computing device can include any number of sensors or components configured to intake or gather data from a user of the electronic computing device including, but not limited to, a camera, a heart rate monitor, a temperature sensor, an accelerometer, a microphone, and a gyroscope. The electronic computing device can also include an input device (e.g., a touchscreen or a keyboard) through which a user may input text and commands.

Further yet in an embodiment of the invention, wherein selecting at least one of the FDS for a user from a plurality of the FDS indicates an assessment of at least one of a feeling, sensation, mood, mental, emotional or physical state of the user. The FDS is assessed based on at least one of a self-reporting questionnaire, standard Pittsburg sleep quality index (PSQI), consensus sleep diary (CSD), daytime insomnia scale (DISS), insomnia symptom questionnaire (ISQ), Pittsburg insomnia rating scale (PIRS), a smartwatch, a ring, a bracelet, a necklace, a body-worn device, headband, waistbelt and IoT devices. Additionally, assessment of the FDS may be based on a combination of self-reporting questionnaires and digital smart devices and/or dermal or supra-dermal sensors. For example, in an embodiment of the invention, the FDS is assessed based on a combination of results from a PSQI and the user's iWatch. The combination of a standard result from a self-reported questionnaire and an electronic device with sensors for example, an iWatch would eliminate the problems with self-report inventories where scores are easily exaggerated or minimized by the person/user completing them. Alternatively, for example, the FDS is assessed and pushed to the user based on a combination of a dermal sensor measuring dopamine and sleep data on a user's iWatch.

Further yet, in an embodiment of the invention, the interaction with the FDS is at least one of a touch, swipe, feel, pull, push, drag, pinch, gesture, multi-touch gestures and natural user interface techniques. Additionally, examples of the natural user interface techniques may include, but are not limited to, eye-tracking, educe, kinetic user interface, intelligent personal assistant, post-WIMP (windows, icons, menus, pointer), scratch input, spatial navigation, tangible user interface, touch user interface, multi-touch, perceptive pixel, Microsoft pixel sense, 3D immersive touch and Xbox Kinect.

In a preferred embodiment of the invention, the guided prompt 102 is at least one of textual, video, audio, visual, gestural, verbal, physical, vibration, manual, phonemic and positional. For example, the system uses the verbal prompt that begins with a sound of bells ringing and the user can interact with the FDS by touching the bells. Alternatively, for example, a textual prompt “find 5 flower pots” may appear and the user touches the flower pots on the screen. Further yet, in an embodiment the method for generating an FDS may further comprise of adjusting at least one of a voice volume, music volume or narration speed of the guided prompt. Alternatively, in an embodiment of the invention, the user may change at least one of the voice volume, music volume or narration speed.

Further yet, in a preferred embodiment of the invention, the digital event is at least one of a meditation, art, color, digital coloring and music to progressively fill the FDS. Moreover, the user may repeat the interactions with the FDS in a steady progressive method to create a digital event, wherein the digital event is at least one of a meditation, art, color, digital coloring and music. The filling of the FDS is progressively tracked until completion either when the FDS is complete or until a time-triggered event. Upon completion, the user is further prompted to complete another FDS to achieve mind integration.

Additionally, in an embodiment of the invention, the completion of the FDS is via a positive affirmation. The positive affirmation is at least one of continuing with another FDS session or based on a time-triggered event. In yet another preferred embodiment, the method of generating an FDS may further comprise establishing a behavioral intervention to target sleep directly or improve a sleep hygiene routine. Sleep is a condition that supports emotional, physiological, and reparative regeneration. It is known to affect a person's quality of life and well-being.

In yet another embodiment of the invention, the time-triggered event is at least one of system event, internal event, timer event, expression event, error event and user event. In accordance with one aspect, generation of reports may occur at regular intervals (such as daily at 12:00 PM, weekly and monthly), on-demand (when the user requests for a report corresponding to the user), when triggered by an event.

Additionally, in an embodiment of the invention, at least one of the FDS pushed is supported by at least one credentialed peer reviewed scientific expert for user review. In a preferred embodiment, an accredited expert or source will require at least two independent sources of peer-reviewed scholarship or data in order to validate the pushing of the FDS to a user.

In another preferred embodiment of the invention, the method for generating the FDS comprises a prescriber to push the FDS, wherein the pushing is at least one of static, dynamic or scheduled based on a user's scheduler criteria. The line of static, dynamic, and, or scheduled messages may be curated by the user, pre-set, or dynamically pushed based on any one of a user parameter. In some embodiments, the timeline module enables the displayed line of static, dynamic, and, or scheduled messages to be further replicated on at least one of a social media timelines or stories.

Further yet, in a preferred embodiment the FDS is pushed based on at least one of a PSQI, CSD, DISS, ISQ, PIRS and smart device generated score. Alternatively, the FDS is selected by the user based at least one of a feeling, sensation, mood, mental, emotional or physical state of the user. In yet another embodiment of the invention, the FDS is pushed based on at least one of a score generated by at least one of the smart-watch, ring, bracelet, necklace, body-worn device, IoT device and a FDS gallery.

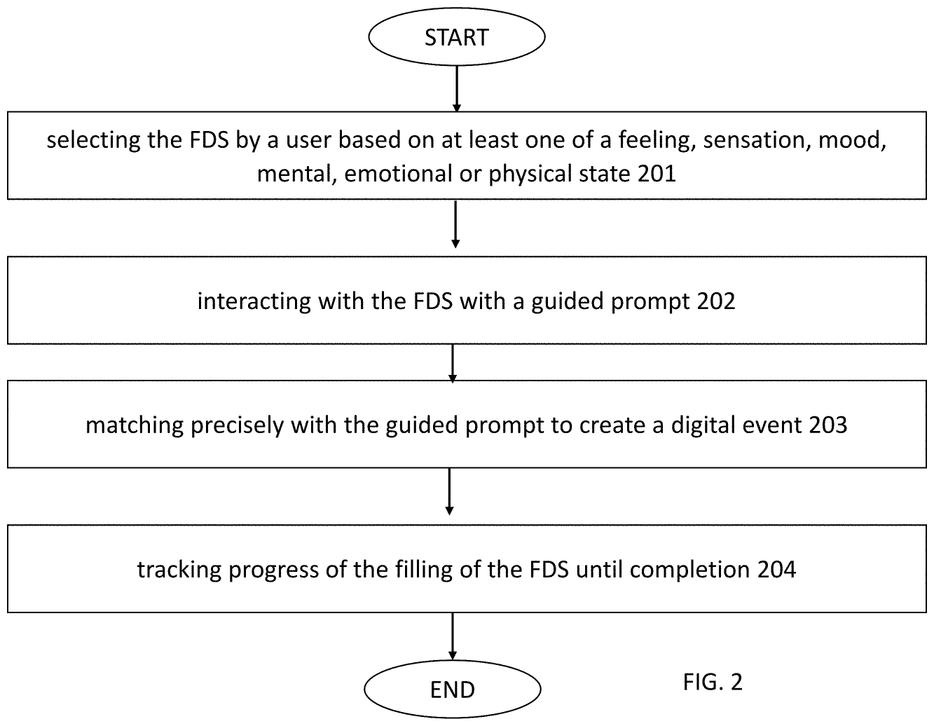

Now in reference to FIG. 2, in an embodiment of the invention, a method for generating a fillable digital scape (FDS) for mind integration comprises the steps of: selecting the FDS by a user based on at least one of a feeling, sensation, mood, mental, emotional or physical state 201, interacting with the FDS with a guided prompt 202, matching precisely with the guided prompt to create a digital event 203 and tracking progress of the filling of the FDS until completion 204.

Further yet, in a preferred embodiment, wherein the interaction with the FDS is at least one of a touch, swipe, feel, pull, push, drag, pinch, gesture, multi-touch gestures and natural user interface techniques. Additionally, examples of the natural user interface techniques may include, but are not limited to, eye-tracking, educe, kinetic user interface, intelligent personal assistant, post-WIMP (windows, icons, menus, pointer), scratch input, spatial navigation, tangible user interface, touch user interface, multi-touch, perceptive pixel, Microsoft pixel sense, 3D immersive touch and Xbox Kinect.

Additionally, in a preferred embodiment of the invention, the guided prompt 202 is at least one of textual, video, audio, visual, gestural, verbal, physical, vibration, phonemic and positional. For example, the system uses the verbal prompt—beginning sound of bells ringing and the user can interact with the FDS by touching the bells. Further yet, in an embodiment the method for generating an FDS may further comprise of adjusting at least one of a voice volume, music volume or narration speed of the guided prompt.

Further yet, in a preferred embodiment of the invention, the digital event is at least one of a meditation, art, color, digital coloring and music to progressively fill the FDS. Additionally, in an embodiment of the invention, the completion of the FDS is via a positive affirmation. The positive affirmation is at least one of continuing with another FDS session or based on a time-triggered event. In yet another preferred embodiment, the method of generating an FDS may further comprise establishing a behavioral intervention to target sleep directly or improve a sleep hygiene routine.

In yet another embodiment of the invention, the time-triggered event is at least one of system event, internal event, timer event, expression event, error event and user event. In accordance with one aspect, generation of reports may occur at regular intervals (such as daily at 12:00 PM, weekly and monthly), on-demand (when the user requests for a report corresponding to the user), when triggered by an event.

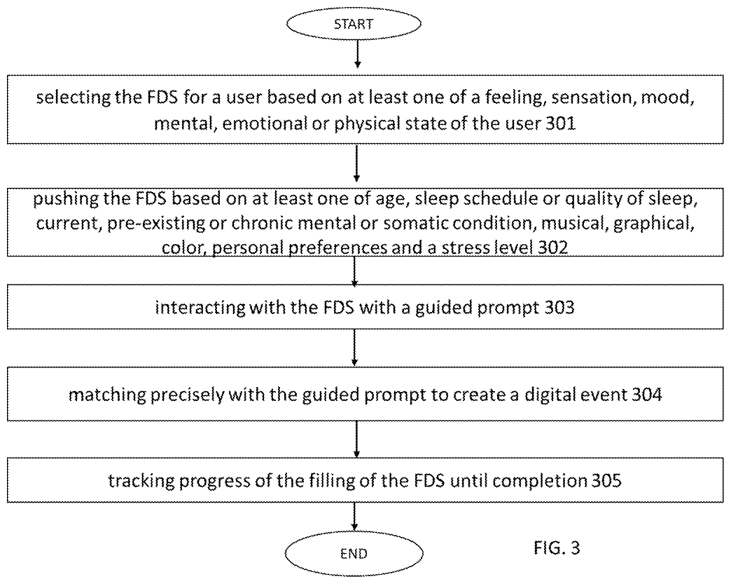

Now with reference to FIG. 3, a method for generating a fillable digital scape (FDS) for mind integration comprises the steps of selecting the FDS for a user based on at least one of a feeling, sensation, mood, mental, emotional or physical state of the user 301, pushing the FDS based on at least one of age, sleep schedule or quality of sleep, current, pre-existing or chronic mental or somatic condition, musical, graphical, color, personal preferences and a stress level 302, interacting with the FDS with a guided prompt 303, matching precisely with the guided prompt to create a digital event 304 and tracking progress of the filling of the FDS until completion 305.

Further yet in an embodiment of the invention, wherein selecting at least one of the FDS for a user from a plurality of the FDS indicates an assessment of at least one of a feeling, sensation, mood, mental, emotional or physical state of the user. The FDS is assessed based on at least one of a self-reporting questionnaire, standard Pittsburg sleep quality index (PSQI), consensus sleep diary (CSD), daytime insomnia scale (DISS), insomnia symptom questionnaire (ISQ), Pittsburg insomnia rating scale (PIRS), a smartwatch, a ring, a bracelet, a necklace, a body-worn device, headband, waistbelt and IoT devices. Additionally, assessment of the FDS may be based on a combination of self-reporting questionnaires and digital smart devices. For example, in an embodiment of the invention, the FDS is assessed based on a combination of results from a PSQI and the user's iWatch. The combination of a standard result from a self-reported questionnaire and an electronic device with sensors for example, an iWatch would eliminate the problems with self-report inventories where scores are easily exaggerated or minimized by the person/user completing them.

Further yet, in an embodiment of the invention, the interaction with the FDS is at least one of a touch, swipe, feel, pull, push, drag, pinch, gesture, multi-touch gestures and natural user interface techniques. Additionally, examples of the natural user interface techniques may include, but are not limited to, eye-tracking, educe, kinetic user interface, intelligent personal assistant, post-WIMP (windows, icons, menus, pointer), scratch input, spatial navigation, tangible user interface, touch user interface, multi-touch, perceptive pixel, Microsoft pixel sense, 3D immersive touch and Xbox Kinect.

In a preferred embodiment of the invention, the guided prompt 302 is at least one of textual, video, audio, visual, gestural, verbal, physical, vibration, manual, phonemic and positional. For example, the system uses the verbal prompt by the sound of bells ringing and the user can interact with the FDS by touching the bells. Further yet, in an embodiment the method for generating an FDS may further comprise of adjusting at least one of a voice volume, music volume or narration speed of the guided prompt.

Further yet, in a preferred embodiment of the invention, the digital event is at least one of a meditation, art, color, digital coloring and music to progressively fill the FDS. Additionally, in an embodiment of the invention, the completion of the FDS is via a positive affirmation. The positive affirmation is at least one of continuing with another FDS session or based on a time-triggered event. In yet another preferred embodiment, the method of generating an FDS may further comprise establishing a behavioral intervention to target sleep directly or improve a sleep hygiene routine. Sleep is a condition that supports emotional, physiological, and reparative regeneration. It is known to affect a person's quality of life and well-being.

Further yet, in an embodiment of the present invention, a method for generating a fillable digital scape (FDS) for mind integration comprises the steps of: selecting the FDS for a user based on at least one of a feeling, sensation, mood, mental, emotional or physical state of the user, pushing the FDS based on at least one of age, sleep schedule or quality of sleep, current, pre-existing or chronic mental or somatic condition, musical, graphical, color, personal preferences and a stress level, interacting with the FDS with a guided prompt, wherein the guided prompt is at least one of textual, video, audio, visual, gestural, verbal, physical, vibration, phonemic and positional, repeating the interactions with the FDS in a steady progressive method to create a digital event, wherein the digital event is at least one of a meditation, art, color, digital coloring and music and tracking progress of the filling of the FDS until completion to achieve mind integration.

Further yet, in an embodiment of the invention, the FDS assessment is based on at least one of a standard Pittsburg sleep quality index (PSQI), consensus sleep diary (CSD), daytime insomnia scale (DISS), insomnia symptom questionnaire (ISQ), Pittsburg insomnia rating scale (PIRS), a smartwatch, a ring, a bracelet, a necklace, a body-worn device and IoT devices.

Additionally, in an embodiment of the invention, the interaction with the FDS is at least one of touch, swipe, feel, pull, push, drag, pinch, gesture, multi-touch gestures and natural user interface techniques. Further yet, the completion of the FDS is via a positive affirmation. The positive affirmation is at least one of continuing with another FDS session or based on a time triggered event. The method of generating the FDS is further comprised of establishing a behavioral intervention to target sleep directly or improve sleep hygiene routine.

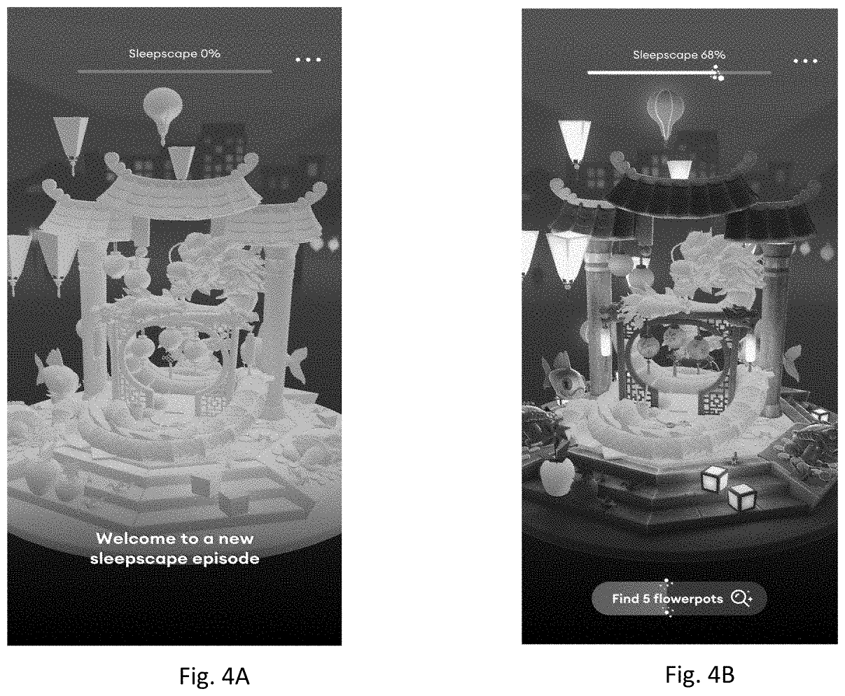

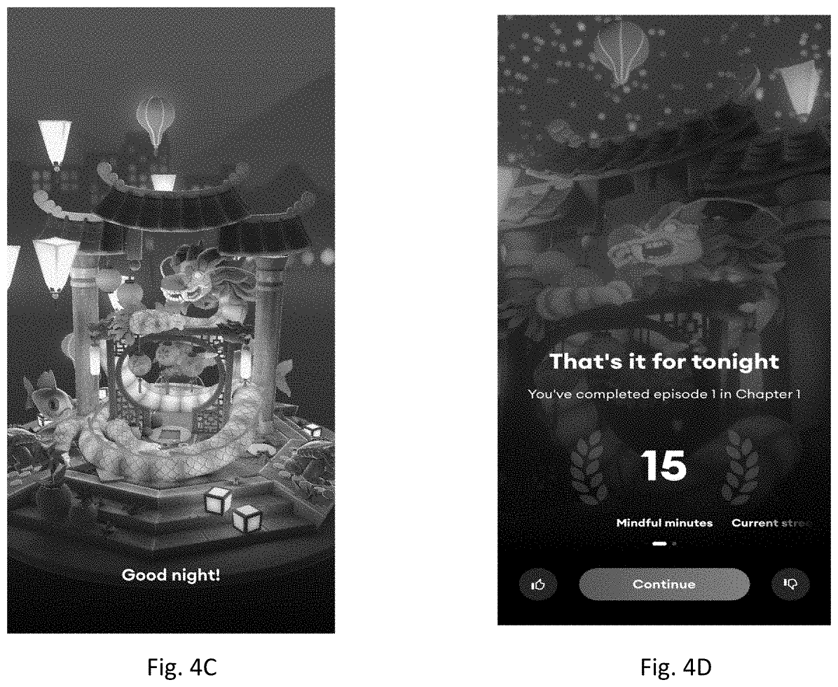

Now with reference to FIG. 4A-C depict representative screen shots of a series of dioramas. Additionally, the FDS is at least one of a digital black & white or a colored, a 2D or a 3D diorama. In an embodiment of the invention, the dioramas are at least one of varying sizes, a miniature or a large-scale object showing a plurality of details. Additionally, the dioramas are a plurality of mountains, oceans, past memories, landscapes, cities, objects, historical events, ecological biomes, cultural scenes, visually depicted literature, ships, buildings, vehicles, railways, architectural and historical venues and entertainment or museums. As seen in FIG. 4A-C, for example, a screen shot of a dragon shrine—unfilled FDS is shown. The FDS is pushed to the user and once the user interacts with the FDS—“find the flowerpots”—guided prompt ‘textual’—on the FDS and matches correctly, a digital event ‘digital color’—“flower pots” are colored yellow and orange, respectively are created. FIG. 4B shows a progressively generation of an FDS. Approximately 68% of the diorama of the dragon shrine is filled with various colors via guided prompts of audio. As shown in FIG. 4C, the generation of the FDS is progressively tracked until completion. FIG. 4C shows the user used the guided prompt—audio and created a digital event—digital color of the dragon shrine to complete the FDS for mind integration. Additionally, a textual prompt—“good night!” is shown in FIG. 4C. that marks completion of the FDS. At this time, the user may choose to complete another FDS to achieve mind integration depending on the users' mental, emotional or physical state. In an embodiment, as shown in FIG. 4D a users has completed one session and have about 15 minutes of mindful minutes for mind integration. A series of FDS may be completed by the user to achieve mind integration.

In another embodiment of the invention, for example, multiple guided prompts—audio and textual may be used to create a digital event. Additionally, the digital event may also be a combination of color and soothing meditative music to achieve mind integration. For example, in FIG. 4A-C (not shown), alternatively multiple guided prompts—audio and textual ‘touch the lanterns and balloons’ are prompted to guide the user to fill the diorama. Further yet, a meditative music may be playing in the background and the coloring of lanterns—yellow and balloons—orange, to create a digital event.

Further yet, in an embodiment of the invention, the dioramas are at least one of static or dynamic. The dynamic or static dioramas may be representations of people, places, things etc. The dynamic dioramas may be able to rotate in all directions 2D while, the static dioramas are stagnant in 2D. Alternatively, the dynamic dioramas may also be able to rotate in all directions in 3D while, the static dioramas may not be able to rotate in any directions in 3D. Further yet in another embodiment of the invention, the dioramas may be dynamic or static in either 2D or 3D.

In yet another preferred embodiment of the invention, the method of generating FDS for mind integration further comprises setting reminders to undergo the FDS intervention on the following day at the same time. Alternatively, depending on the user's mental, physical and emotional state, the user may set multiple reminders to complete FDS the following day at the same time. Additionally, the user may set multiple reminders weekly, biweekly or monthly to complete FDS based on the emotional, mental and physical status of the user. Alternatively, the user may set multiple reminders at multiple times on the day at any given time to achieve mind integration depending on the emotional, mental and physical state. Additionally, in an embodiment of the invention, the method further comprises of setting reminders to undergo the FDS sessions as per the user's needs. Further yet, the user may repeat a plurality of the FDS sessions until mind integration or sleep is achieved.

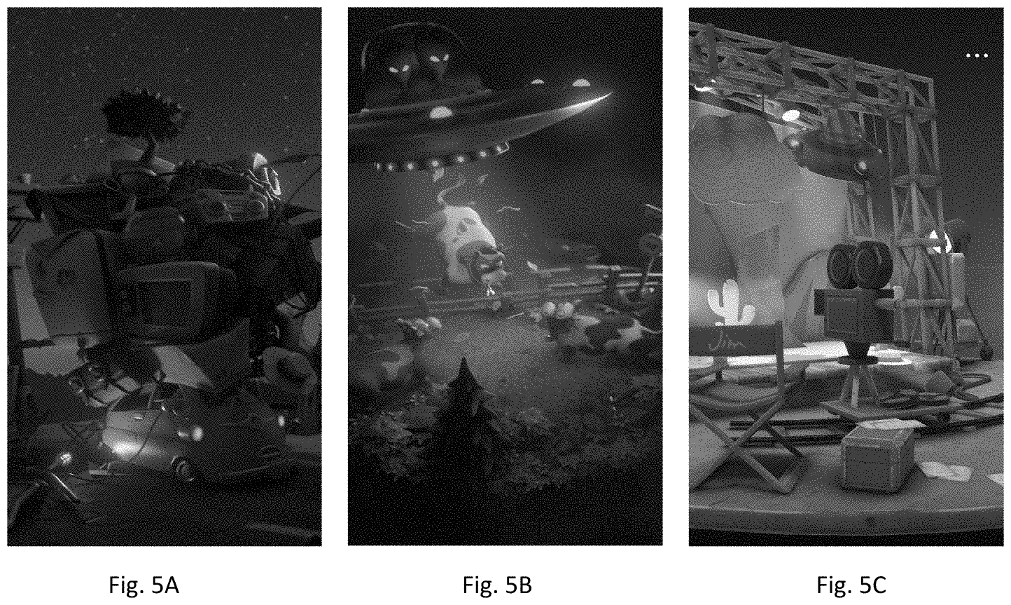

Now with reference to FIG. 5A-C depict representative screen shots of a series of dioramas. Additionally, the FDS is at least one of a digital black & white or a colored, a 2D or a 3D diorama. FIG. 5A-C shows a representative example of a series of dioramas in sequence in an episodic format showcasing a story of a guy named Jim making a cross country move from New York to the West coast and the adventures of his journey. For example, FIG. 5A shows Jim's stuff packed and ready to move to the west coast, FIG. 5B., shows Jim encountering aliens that are trying to abduct a cow, FIG. 5C represents Jim directing a film in LA about his encounter and witnessing the cow theft. These scenarios in the series of episodic FDS creates a bond between the user and the characters, thus creating a sense of belonging, comfort, structure and predictability which are the main attributes towards generating mind integration.

The dynamic or static dioramas may be representations of people, places, things etc. The dynamic dioramas may be able to rotate in all directions 2D while, the static dioramas are stagnant in 2D. Alternatively, the dynamic dioramas may also be able to rotate in all directions in 3D while, the static dioramas may not be able to rotate in any directions in 3D. Further yet in another embodiment of the invention, the dioramas may be dynamic or static in either 2D or 3D.

In yet another embodiment of the invention, the FDS is further based on input from at least one of the users, a user profile or crawled data. Other user profile or demographic information may further inform the prescribers choice of message type, such as age, education level, voting preference, etc. User profile or demographic information may be user inputted or digitally crawled.

Further yet, in an embodiment of the invention, the method for generating the FDS further comprises a prescriber to push the FDS. The pushing of the FDS is at least one of static, dynamic or scheduled based on a user's schedular criteria. The dioramas are at least one of static or dynamic. Alternatively, the prescriber's choice of FDS may not be specific to a user, user profile, or crawled user data. Additionally, in an embodiment of the invention, wherein the FDS is further based on a user history, wherein the user history is the users tracked level of engagement with any previous FDS. Further yet, in a certain embodiment, the prescriber may have to choose between a plurality of FDS(s). This type of FDS assignment may be completely arbitrary. In other embodiments, the FDS assignment may be not specific to a user-generated or crawled profile but may be based on user history. In other words, a user's tracked level of engagement with a previous FDS or FDS from a previous session may inform FDS assignment by the prescriber. Tracking engagement of a user with a pushed or prescribed therapeutic FDS may be by camera-captured eye gazing, touch-screen interaction, time span between pushed therapeutic and user follow-up action, choice of follow-up action, etc.

In some embodiments, the full list of FDS(s) is not grouped by FDS type or along any design categories, but rather simply listed arbitrarily and mapped or matched to an appropriate FDS type. In this arbitrarily listed manner, the prescriber may match to more than one FDS type. Likewise, a user may be defined by more than one FDS type and be prescribed the same FDS type.

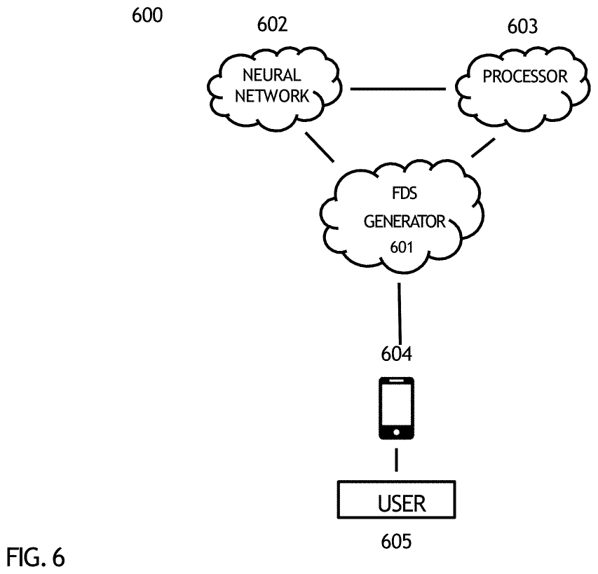

Now with reference to FIG. 6, depicts a schematic of a system for generating a fillable digital scape (FDS) to reinforce mind integration. In one embodiment, a system can include: FDS generator 601, a processor 603, and an electronic computing device 604 associated with a particular user 605. The FDS generator 601, the processor 603, and the electronic computing device 604 are communicatively coupled via a communication network. The network may be any class of wired or wireless network including any software, hardware, or computer applications that can provide a medium to exchange signals or data. The network may be a local, regional, or global communication network.

The electronic computing device 604 may be any electronic device capable of sending, receiving, and processing information. Examples of the computing device include, but are not limited to, a smartphone, a mobile device/phone, a Personal Digital Assistant (PDA), a computer, a workstation, a notebook, a mainframe computer, a laptop, a tablet, a smart watch, an internet appliance and any equivalent device capable of processing, sending and receiving data. The electronic computing device 604 can include any number of sensors or components configured to intake or gather data from a user of the electronic computing device 604 including, but not limited to, a camera, a heart rate monitor, a temperature sensor, an accelerometer, a microphone, and a gyroscope. The electronic computing device 604 can also include an input device (e.g., a touchscreen or a keyboard) through which a user may input text and commands.

In one embodiment, the system 600 may receive an electronic message in the form of an electroencephalograph (EEG) output. For example, in this embodiment, a user 605 can generate a message using an electronic device communicatively coupled to the user 605 and capable of performing an electroencephalograph to measure and record the electrochemical activity in the user's brain. In this example, the system 600 can transcribe the EEG output into an electronic message or receive a transcription of the EEG output from the electronic device communicatively coupled to the user 605. After receiving or generating the electronic message from the EEG, the FDS generator 605 can then analyze the message content within the electronic message, determine the mood of the message content, and apply a corresponding FDS to the electronic message. The message may be at least one of age, sleep schedule or quality of sleep, current, pre-existing or chronic mental or somatic condition, musical, graphical, color, personal preferences and a stress level.

In one example of this embodiment, a user 605 is connected to an augmented reality (AR) or virtual reality (VR) headset capable of performing an EEG or an equivalent brain mapping technique. The user 605 can generate a message simply by thinking of what the user 605 is feeling or would like to say. The headset can monitor and record these thoughts and feelings using the EEG and transcribe the thoughts and feelings into an electronic message or send the EEG output signals directly to the system 600. The system 600 can then analyze the message content included within the electronic message, determine the mood or sentiment of the message content, and apply a corresponding FDS to the electronic message, creating a vectorized FDS. The system 600 can then send the vectorized FDS to the user 605 to reinforce mind integration.

In an embodiment, the neural network 602 can employ machine learning techniques to analyze this database for patterns and trends in order to dynamically improve the performance of the FDS generator 601. For example, the neural network 602 may determine through the application of an algorithm that the user 605 prefers the “sound of chirping birds” ten times more than the “sound of falling rain” to calm the mind. Thus, even though the “sound of chirping birds” may bring about less of a positive impact than “sound of rain” for the average user or person, the neural network can determine that, for the particular user 605, “chirping birds” generally carries more emotional weight than the “sound of falling rain.” In this example, the neural network 602 can then update the processor 603 and FDS generator 601 and the library of emotionally charged language accordingly. Examples of machine learning may be at least one of a convolution neural network, associated model, training data set, feed-forward neural network, and, or back-propagated neural network.

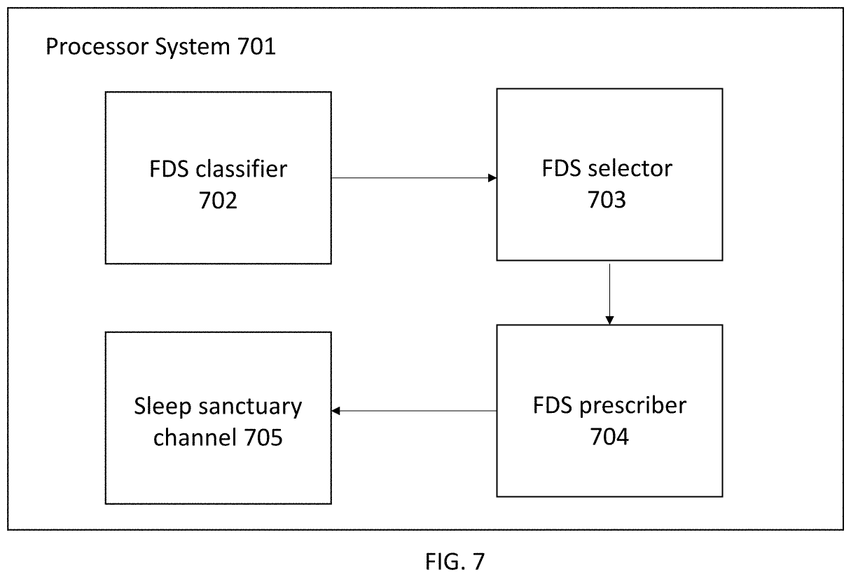

Now with reference to FIG. 7, a system for generating a fillable digital scape (FDS) to reinforcing mind integration comprising of, a FDS classifier 702, a FDS selector 703, a FDS prescriber 704, a non-transitory storage element coupled to the processor 701, encoded instructions stored in the non-transitory storage element, wherein the encoded instructions when implemented by the processor 701, configure the sleep sanctuary channel 705 to: classify the FDS based on at least one of age, sleep schedule or quality of sleep, current, pre-existing or chronic mental or somatic condition, musical, graphical, color, personal preferences and a stress level by the FDS classifier 702, select at least one of the FDS for the user from a plurality of the FDS by assessing at least one of a feeling, sensation, mood, mental, emotional or physical state of the user via the FDS selector 703, push the FDS via the FDS prescriber 704 personalized to the user based on at least one stored message coupled to the FDS, wherein said message comprises of a guided prompt to interact with the FDS to create a digital event, wherein the digital event is at least one of a meditation, art, color, digital coloring and music to progressively fill the FDS and track the progress of the filling of the FDS until completion.

Still in continuing reference to FIG. 7, a system depicted as a block diagram, wherein the processing system (701) and modules (702-705) are specifically interrelated and configured to perform a particular sub-routine in accordance with at least one of a defined logic, probabilistic learning (machine learning/AI), statistical modeling, or rules, in order to achieve mind integration. In one embodiment, the user may select the FDS or the system may push the FDS and select the content type and action type of the FDS. Examples of content type may be video, music, film clip, GIF, photo, PDF, screen shot, social media post, text message template, VR asset, AR asset in the form of a 2D or 3D diorama. In some embodiments, a user may only need to choose the treatment/condition FDS type.

In continuing reference to FIG. 7, the content type and action type may be autonomously generated without user input or data. The FDS classifier 702 may take multiple bound-boxed crops from at least one of a 2D or 3D parsed or non-parsed image frame, perform object or event detection, and then join crops to form a mask for the original image. The reconstructed mask or loose crops are then stitched together and based on at least one of an object detected, facial feature, overall context, emotional cues, stylistic elements, deconstructed text and, or audio, at least one condition/FDS from a plurality of conditions/FDS is selected by the FDS selector 703, said selected FDS indicating at least one of a feeling, sensation, mood, mental state, physical state, emotional condition, physical status. Once the appropriate condition/FDS is selected, the FDS prescriber 704 will assign an action label to the digital content corresponding to the selected action by the prescriber based on a severity-graded look-up guide.

In other embodiments, the FDS prescriber 704 may be used to do at least one of a content review, action/FDS selection. Assigning a second message to the same user in the same session for the first defined FDS type. The FDS prescriber 604 may perform at least one of a content review, action/FDS selection overlay in a logic-defined or rule-based manner, wherein gathered, contextualized, or profiled data may further inform at least one of the content review, action/FDS selection, and overlay.

For instance, when a system-generated FDS is selected as ‘In the Wilderness’ for user A, a FDS prescriber 704 assigns an action label (chirping cricket sounds in the woods and inspiring message to stabilize mood and provide stability for happiness to flourish and to calm the mind to achieve sleep). In a another embodiment, the action label may also be tapped for providing additional information, such as drug/neurotransmitter information, benefits, and citations. In one embodiment, a secondary prescriber (not shown) may push a subsequent message or content, such as a meditative music, immediately upon a touch interaction with the first inspiring message pushed. In other embodiments, a level of engagement, interaction or compliance may be tracked by the system to infer severity of the FDS. For instance, if user A does not comply with the touch-interaction requests from the first inspiring message or meditative music recommendation of the second message, then the secondary prescriber (not shown) may push another action, such as deep breathing to achieve mind integration. For the purposes of inferring severity of FDS, any number of diagnostics that leverage any one of the on-device tools may be used, such as gyroscopic sensors or cameras. Severity may also be inferred from contextual data gathered from off-board devices, lot objects, crawled social media data, etc.

In one embodiment, action/engagement with the FDS of the digital content may be based on learned user history, such as previous liked history and, or engagement/reaction (compliance/non-compliance) to receiving an action/content based FDS. Based on such history of action and, or engagement, a FDS prescriber 704 may assign an action label for uploaded content by user A that is consistent or departed from the previous action/engagement.

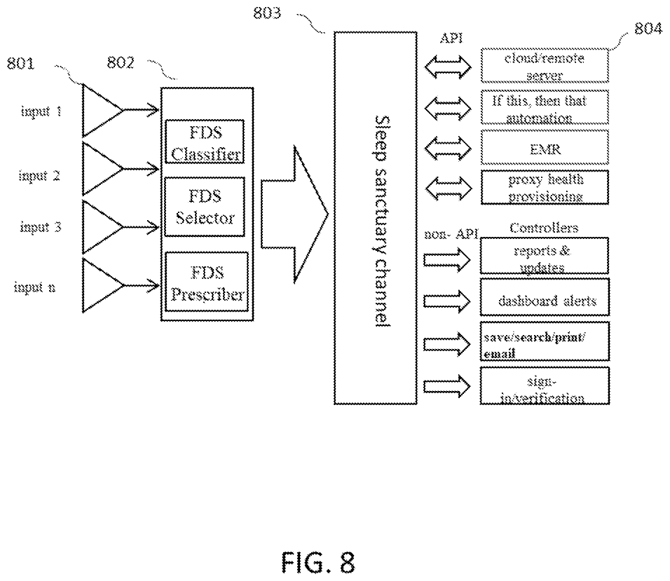

FIG. 8 is a representative interaction flow of the FDS generating system in accordance with an aspect of the invention. In a preferred embodiment of the invention, the inputs 801 recognizes a command and processes input from anyone of a user's device or user, wherein the input is any one of a digital content uploaded from a user. The digital content uploaded by the user is originated from at least one of a stored, received, visited, curated, and created source. Furthermore, the content may be at least one of saved, processed, edited, and uploaded in edited form; or uploaded in original/received form; and forwarded to the downstream system that provides the recognized command for enabling FDS generating system of the digital content.

In an embodiment of the invention, the inputs 801 may be motion characteristics corresponding to at least one of, physical activity, physiological and sleep related characteristics of a user quantified from a body worn or user device. Additionally, inputs 801 may account for environmental conditions, such as wind velocity, temperature, humidity, aridness, light, darkness, noise pollution, exposure to UV, airborne pollution and radioactivity quantified from a body-worn/user device and, or remote stations. Further yet, data generated from a periodic survey pushed to a body worn/user device may be used to generate a behavioral profile of the user, which may serve as an input 801 or inform an input 801. The system may flag a threshold discrepancy between a composite behavioral profile and a reference behavioral profile to detect or select an appropriate FDS, in addition to the parsed digital content by the FDS classifier 802, FDS selector 802, FDS prescriber 802, whereby the appropriate FDS is determined by machine learning algorithms to trigger a number of downstream provisioning(s) 804.

Further yet, in another embodiment, the system may further comprise integration with any one of a third-party application via an Application Program Interface (API) 804. This allows for 3rd party database integration, such as Electronic Medical Records (EMR), health monitoring, proxy health provisioning, remote server and, or a cloud-based server for other downstream analytics and provisioning. Additionally, the completed automated responses may be saved onto a remote cloud-based server for easy access for data acquisition and archival analytics for future use.

In another embodiment of the invention, the system may allow for easy saving, searching, printing, and sharing of completed automated response information with authorized participants. Additionally, the system may allow for non-API applications, for example, building reports and updates, create dashboard alerts as well as sign in/verifications 804. Alternatively, sharing may be possible with less discrimination based on select privacy filters. Moreover, the system may be integrated with certain workflow automation tools, prompting the system to perform a task command, provided a trigger is activated based on the threshold discrepancy. In an embodiment of the invention, at least one conditional event triggers at least one action controlled by a “if this, then that” 804 script manager. Further yet, the “if this, then that” 804 script manager is embedded with an “and, or” trigger or action operators, allowing increased triggers or actions in a command set.

In another instance, the script manager may be embedded with a “if, this, then that” as well as a “and, or” trigger or action operator for increased triggers either downstream or upstream of a command set. While not shown in FIG. 8, “IF” a user uploads content/action with an FDS rating of sad, “THEN”, the user will be sent prescriptive content to counter the sadness, such as uplifting music “AND” the users closest friend will receive an email/text reminder to get in touch with the user. All of the commands are automatically triggered once an “IF” conditional event is reached.

In yet another embodiment of the invention, “OR” operators may be used instead of the “AND” operator. Further, any number of “AND” and, or “OR” operator may be used in a command function. Such an automation layer may add further efficiencies. An ecosystem of apps may provide for an API-mediated link to the system for enhanced co-interactivity among user's network, diagnostics, and other measurables.

The processer system 802 may further be communicatively coupled to at least one of a sleep sanctuary channel 803, interface module, display module, input module, logic module, a context module, timeline module, tracking module, notification module, payment/gifting module, and marketplace module in order to effectuate any number of remote provisioning. In accordance with one aspect, the notification module may be configured to generate reports at regular intervals (such as daily at 12:00 PM, weekly and monthly), on-demand (when the user requests for a report corresponding to the user), when triggered by an event, or upon a detected severity. In an embodiment of the present invention, the notification module may also be configured to send a notification to the user or to a chosen loved one of the users. The notification may be a message, a phone call or any other communication means.

In an embodiment of the present invention, a timeline module may push already pushed messages in at least one of a static, dynamic, and, or scheduled fashion based on at least one of the user's scheduler criteria. The line of static, dynamic, and, or scheduled messages may be curated by the user, pre-set, or dynamically pushed based on any one of a user parameter. In some embodiments, the timeline module enables the displayed line of static, dynamic, and, or scheduled messages to be further replicated on at least one of a social media timelines or stories. In other words, the timeline module enables the displayed messages to be further shared with social media outlets.

In an embodiment of the present invention, a payment or gifting module may enable purchasing and gifting donations, physical objects, or digital assets. In an embodiment of the present invention, a marketplace module may enable purchasing digital assets. The gifting and marketplace module may further be coupled to a distributive digital ledger, wherein each transaction among any user is represented as a unique node in the digital ledger. Each node tagged with meta data facilitating at least one of a transaction, validation and, or registration for each transaction.

Embodiments are described at least in part herein with reference to flowchart illustrations and/or block diagrams of methods, systems, and computer program products and data structures according to embodiments of the disclosure. It will be understood that each block of the illustrations, and combinations of blocks, can be implemented by computer program instructions. These computer program instructions may be provided to a processor of a general-purpose computer, special purpose computer, or other programmable data processing apparatus to produce a machine such that the instructions, which execute via the processor of the computer or other programmable data processing apparatus, create means for implementing the functions/acts specified in the block or blocks.

These computer program instructions may also be stored in a computer-readable memory that can direct a computer or other programmable data processing apparatus to function in a particular manner such that the instructions stored in the computer-readable memory produce an article of manufacture including instruction means which implement the function/act specified in the block or blocks.

The computer program instructions may also be loaded onto a computer or other programmable data processing apparatus to cause a series of operational steps to be performed on the computer or other programmable apparatus, to produce a computer implemented process such that, the instructions which execute on the computer or other programmable apparatus provide steps for implementing the functions/acts specified in the block or blocks. In general, the word “module” as used herein, refers to logic embodied in hardware or firmware, or to a collection of software instructions, written in a programming language, such as, Java, C, etc. One or more software instructions in the unit may be embedded in firmware. The modules described herein may be implemented as either software and/or hardware modules and may be stored in any type of non-transitory computer-readable medium or other non-transitory storage elements. Some non-limiting examples of non-transitory computer-readable media include CDs, DVDs, BLU-RAY, flash memory, mobile device, remote device, and hard disk drives.

We claim:

1. A method for generating a fillable digital scape (FDS) for mind integration, said method comprising the steps of:
pushing the FDS;
interacting with the FDS with a guided prompt;
matching precisely with the guided prompt to create a digital event; and
tracking progress of the filling of the FDS until completion.

2. The method of claim 1, wherein pushing the FDS is based on at least one of age, sleep schedule or quality of sleep, current, pre-existing or chronic mental or somatic condition, musical, graphical, color, personal preferences and a stress level.

3. The method of claim 1, wherein selecting at least one of the FDS for a user from a plurality of said FDS, the FDS indicating an assessment of at least one of a feeling, sensation, mood, mental, emotional or physical state of the user.

4. The method of claim 3, wherein the FDS assessment is based on at least one of a standard Pittsburg sleep quality index (PSQI), consensus sleep diary (CSD), daytime insomnia scale (DISS), insomnia symptom questionnaire (ISQ), Pittsburg insomnia rating scale (PIRS), a smartwatch, a ring, a bracelet, a necklace, a body-worn device and IoT devices.

5. The method of claim 1, wherein the interaction with the FDS is at least one of a touch, swipe, feel, pull, push, drag, pinch, gesture, multi-touch gestures and natural user interface techniques.

6. The method of claim 1, wherein the guided prompt is at least one of textual, video, audio, visual, gestural, verbal, physical, vibration, manual, phonemic and positional.

7. The method of claim 1, wherein the digital event is at least one of a meditation, art, color, digital coloring and music to progressively fill the FDS.

8. The method of claim 1, wherein the completion of the FDS is via a positive affirmation.

9. The method of claim 8, wherein the positive affirmation is at least one of continuing with another FDS session or based on a time-triggered event.

10. The method of claim 9, wherein the time-triggered event is at least one of system event, internal event, timer event, expression event, error event and user event.

11. The method of claim 1, further comprising adjusting at least one of a voice volume, music volume or narration speed of the guided prompt.

12. The method of claim 1, further comprising establishing a behavioral intervention to target sleep directly or improve a sleep hygiene routine.

13. The method of claim 1, wherein at least one of the FDS pushed is supported by at least one credentialed peer reviewed scientific expert for user review.

14. The method of claim 1, further comprising a prescriber to push the FDS, wherein the pushing is at least one of static, dynamic or scheduled based on a user's scheduler criteria.

15. The method of claim 1, wherein the FDS is pushed based on at least one of a PSQI, CSD, DISS, ISQ, PIRS and smart device generated score.

16. The method of claim 1, wherein the FDS is pushed based on at least one of a score generated by at least one of the smart-watch, ring, bracelet, necklace, body-worn device, IoT device and a FDS gallery.

17. The method of claim 1, wherein the FDS is at least one of a digital black & white or a colored, a 2D or a 3D diorama.

18. The method of claim 17, wherein the dioramas are at least one of varying sizes, a miniature or a large-scale object showing a plurality of details.

19. The method of claim 17, wherein the dioramas are a plurality of mountains, oceans, past memories, landscapes, cities, objects, historical events, ecological biomes, cultural scenes, visually depicted literature, ships, buildings, vehicles, railways, architectural and historical venues and entertainment or museums.

20. The method of claim 17, wherein the dioramas are at least one of static or dynamic.

21. The method of claim 1, wherein the FDS is further based on input from at least one of the user, a user profile or crawled data.

22. The method of claim 1, wherein the FDS is further based on a user history, wherein the user history is the users tracked level of engagement with any previous FDS.

23. The method of claim 1, further comprising setting reminders to undergo the FDS intervention on the following day at the same time.

24. The method of claim 1, further comprising setting reminders to undergo the FDS sessions as per the user's needs.

25. The method of claim 1, wherein the user repeats a plurality of the FDS sessions until mind integration or sleep is achieved.

26. The method of claim 1, wherein the FDS are assigned and pushed by machine learning aspects.

27. The method of claim 26, wherein the machine learning aspect is at least one of a neural network, deep learning method, and support vector machine.

28. The method of claim 1, wherein said FDS are shared to others in network or out of network.

29. A method for generating a fillable digital scape (FDS) for mind integration, said method comprising the steps of:
selecting the FDS by a user based on at least one of a feeling, sensation, mood, mental, emotional or physical state;
interacting with the FDS with a guided prompt;
matching precisely with the guided prompt to create a digital event; and
tracking progress of the filling of the FDS until completion.

30. The method of claim 29, wherein the interaction with the FDS is at least one of a touch, swipe, feel, pull, push, drag, pinch, gesture, multi-touch gestures and natural user interface techniques.

31. The method of claim 29, wherein the guided prompt is at least one of textual, video, audio, visual, gestural, verbal, physical, vibration, phonemic and positional.

32. The method of claim 29, wherein the digital event is at least one of a meditation, art, color, digital coloring and music to progressively fill the FDS.

33. The method of claim 29, wherein the completion of the FDS is via a positive affirmation.

34. The method of claim 33, wherein the positive affirmation is at least one of continuing with another FDS session or based on a time-triggered event.

35. The method of claim 1, further comprising establishing a behavioral intervention to target sleep directly or improve a sleep hygiene routine.

36. A method for generating a fillable digital scape (FDS) for mind integration, said method comprising the steps of:
selecting the FDS for a user based on at least one of a feeling, sensation, mood, mental, emotional or physical state of the user;
pushing the FDS based on at least one of age, sleep schedule or quality of sleep, current, pre-existing or chronic mental or somatic condition, musical, graphical, color, personal preferences and a stress level;
interacting with the FDS with a guided prompt;
matching precisely with the guided prompt to create a digital event; and
tracking progress of the filling of the FDS until completion.

37. The method of claim 36, wherein the FDS assessment is based on at least one of a standard Pittsburg sleep quality index (PSQI), consensus sleep diary (CSD), daytime insomnia scale (DISS), insomnia symptom questionnaire (ISQ), Pittsburg insomnia rating scale (PIRS), a smartwatch, a ring, a bracelet, a necklace, a body-worn device and IoT devices.

38. The method of claim 36, wherein the interaction with the FDS is at least one of touch, swipe, feel, pull, push, drag, pinch, gesture, multi-touch gestures and natural user interface techniques.

39. The method of claim 36, wherein the guided prompt is at least one of textual, video, audio, visual, gestural, verbal, physical, vibration, phonemic and positional.

40. The method of claim 36, wherein the digital event is at least one of a meditation, art, color, digital coloring and music to progressively fill the FDS.

41. The method of claim 36, wherein the completion of the FDS is via a positive affirmation.

42. The method of claim 41, wherein the positive affirmation is at least one of continuing with another FDS session or based on a time-triggered event.

43. The method of claim 36, further comprising establishing a behavioral intervention to target sleep directly or improve a sleep hygiene routine.

44. A method for generating a fillable digital scape (FDS) for mind integration, said method comprising the steps of:
selecting the FDS for a user based on at least one of a feeling, sensation, mood, mental, emotional or physical state of the user;
pushing the FDS based on at least one of age, sleep schedule or quality of sleep, current, pre-existing or chronic mental or somatic condition, musical, graphical, color, personal preferences and a stress level;
interacting with the FDS with a guided prompt, wherein the guided prompt is at least one of textual, video, audio, visual, gestural, verbal, physical, vibration, phonemic and positional;
repeating the interactions with the FDS in a steady progressive method to create a digital event, wherein the digital event is at least one of a meditation, art, color, digital coloring and music; and
tracking progress of the filling of the FDS until completion to achieve mind integration.

45. The method of claim 44, wherein the FDS assessment is based on at least one of a standard Pittsburg sleep quality index (PSQI), consensus sleep diary (CSD), daytime insomnia scale (DISS), insomnia symptom questionnaire (ISQ), Pittsburg insomnia rating scale (PIRS), a smartwatch, a ring, a bracelet, a necklace, a body-worn device and IoT devices.

46. The method of claim 44, wherein the interaction with the FDS is at least one of touch, swipe, feel, pull, push, drag, pinch, gesture, multi-touch gestures and natural user interface techniques.

47. The method of claim 44, wherein the completion of the FDS is via a positive affirmation.

48. The method of claim 47, wherein the positive affirmation is at least one of continuing with another FDS session or based on a time-triggered event.

49. The method of claim 44, further comprising establishing a behavioral intervention to target sleep directly or improve a sleep hygiene routine.
